(19) United States
(12) Patent Application Publication
(10) Pub. No.: US 2025/0333791 A1
(43) Pub. Date: Oct. 30, 2025

(54) DETERMINATION OF THE RISK OF DEATH OF A PATIENT INFECTED BY A RESPIRATORY VIRUS BY MEASURING THE EXPRESSION LEVEL OF THE ADGRE3 GENE

(71) Applicants: bioMérieux, Marcy-l'Étoile (FR); Hospices Civils de Lyon, Lyon (FR); Universite Claude Bernard Lyon 1, Villeurbanne (FR)

(72) Inventors: Claire TARDIVEAU, Saint Germain au Mont d'Or (FR); Anne-Claire LUKASZEWICZ, Lyon (FR); Guillaume MONNERET, Lyon (FR); Fabienne VENET, Lyon (FR)

(21) Appl. No.: 18/880,034

(22) PCT Filed: Jul. 5, 2023
(86) PCT No.: PCT/EP2023/068506

§ 371 Date: Dec. 30, 2024

(30) Foreign Application Priority Data
Jul. 6, 2022 (EP) 22183391.6

(51) Int. Cl. C12Q 1/6883

(52) U.S. Cl. CPC C12Q 1/6883; C12Q 2600/118; C12Q 2600/158

(57) ABSTRACT

The invention relates to an in vitro or ex vivo determination method for determining the risk of death of a patient infected by a respiratory virus, said method comprising the steps of measuring, in a biological sample from said patient, the expression level of the ADGRE3 gene, and of comparing the expression level thus measured or a value derived from this quantity with a predetermined reference value. The method thus makes it possible to conclude that there is an increased risk of death of the patient when an underexpression of the ADGRE3 gene is statistically demonstrated from the biological sample. Advantageously, the measurement of the expression level of ADGRE3 can be supplemented by the measurement of one or more additional genes such as C3AR1, CD177, OAS2, CIITA, IL-10, IL1R2, CD74, TDRD9) and combinations thereof. The invention also relates to kits for measuring the expression of ADGRE3 and optionally of one or more additional genes.

### TECHNICAL FIELD

The present invention relates to the technical field of methods and kits for in vitro diagnostics. In particular, the invention relates to methods and kits that make it possible to determine the presence of an increased risk of death of a subject infected with a respiratory virus, particularly with a respiratory virus such as SARS-COV-2 or a variant thereof.

### PRIOR ART

To date, the coronavirus (COVID-19) pandemic caused by the severe acute respiratory syndrome coronavirus 2 (SARS-COV-2) has infected more than 550 million patients globally and caused more than 6.3 million deaths.

The severity of the disease varies considerably from one patient to another, and the majority of patients are asymptomatic or exhibit minimal symptoms such as fever, a cough and/or shortness of breath. Nevertheless, 5 to 10% of patients require intensive care due to the rapid evolution (9 to 12 days) to a more severe or critical form with the development, for example, of acute respiratory distress syndrome (ARDS) and/or sever hypoxemia, acute pulmonary lesions (APL), multiple organ failure, and even death (C. Huang et al., 2020).

Immune response is of critical importance in the physiopathology of COVID-19, and the most severe phenotype of infected patients on admission to intensive care is characterized by a complex immune profile which evolves over time (E. Z. Ong et al., 2020).

In spite of everything, in patients suffering from severe COVID-19, the immune response can be defined by an alteration to inflammatory and immune responses with pronounced lymphopenia, a high neutrophil and monocyte count, a decrease in monocyte HLA-DR expression, a moderate plasma cytokine storm, inadequate type-I interferon signaling and down-regulation of interferon-stimulated genes (ISG) (F. Venet et al., Crit Care, 2021). These alterations can lead to microthrombosis and to tissue lesions, ultimately leading to ARDS, multiple organ failure and death (Hadjadj J et al., 2020).

During the pandemic, numerous exploratory studies were performed in order to understand the immune processes involved in COVID-19. Overall, these studies used various mixed flow cytometry approaches (spectral flow cytometry, multicolor flow cytometry, time-of-flight mass spectrometry), functional tests, and also multiplex analysis of soluble mediators. The results were mainly analyzed using multidata/multiomics approaches. While they provide crucial information regarding the physiopathology of COVID-19, these approaches are mainly based on clinical research tools which cannot be used in routine clinical practice at a patient's bedside or in a central laboratory for characterizing the immune profile and thus the potential risk of death, due to the large number of limitations: significant implementation time, lack of standardization, low level of reproducibility across cohorts, and substantial cost.

Consequently, there is a need to develop alternative approaches to make it possible to effectively predict or identify the mortality risk of patients infected with respiratory viruses such as those responsible for COVID-19, more quickly and at the patient's bedside or in a central laboratory. In this regard, the measurement of biomarker(s), particularly transcriptomic biomarker(s), is a potential avenue which is being constantly explored.

In this context, it has in particular been established that the longitudinal trajectories of 11 circulating immunity-based biomarkers could be associated with patients' mortality when they were increased (10) or decreased (1), thereby providing initial evidence that immunity-based biomarkers could make it possible to obtain an early warning of the outcome for patients suffering from COVID-19 (Abers et al., 2021).

Gene expression profiles have also been described for predicting the outcome for patients suffering from COVID-19 (Guardela B et al., 2021).

Thus, the biomarker CD177 has been described as possibly being associated with severity and the risk of death of patients suffering from COVID-19. Particularly, the stability of CD177 protein levels in serum from patients suffering from severe COVID-19 over the course of the disease is described as being a sign of a less favourable prognosis which could lead to death (Levy Y et al., 2021).

Nevertheless, in light of the global prevalence, it is still necessary to identify new biomarkers in order to supplement the clinician's arsenal with other alternatives which make it possible to effectively predict or identify the risk of death of subjects infected with respiratory viruses, particularly those responsible for COVID-19, in order to be able to adapt treatment, preferentially early on, through guided therapies, and thereby improve those subjects' chances of survival.

### SUMMARY

A first subject of the invention relates to an in vitro or ex vivo method for determining the risk of death of a subject infected with a respiratory virus, said method comprising a step of measuring the expression level of the ADGRE3 gene in a biological sample from said subject, followed by a step of comparing the expression level of the ADGRE3 gene thus measured, particularly at the mRNA level, or a value derived from said level, with a predetermined reference value.

On the basis of the result of the comparison, a conclusion that there is an increased risk of death of said subject is drawn when a decrease in the expression of the ADGRE3 gene is identified.

Advantageously, the reference value corresponds to the average ADGRE3 expression level obtained from biological samples originating from a population of subjects who are not infected with any respiratory virus, or to the average ADGRE3 expression level obtained from biological samples originating from a population of subjects who are infected with a respiratory virus and who are known to have survived following infection, particularly in the 28 days following admission to a healthcare facility or in the 37 days post-infection.

In a preferred embodiment, the respiratory virus is SARS-COV-2 or a variant thereof.

Advantageously, the performance of determining the risk of death can be improved by measuring the expression level of additional genes. Thus, according to a particular embodiment, the method also comprises a step of measuring the expression level of at least one additional gene selected from C3AR1, CD177, OAS2, CIITA, IL-10, IL1R2, CD74, TDRD9 and combinations thereof, preferably selected from C3AR1, CD177, OAS2, CIITA, IL-10, IL1R2 and combinations thereof, and more preferably selected from C3AR1, OAS2, CIITA, IL-10 and combinations thereof, in the biological sample from said subject.

The measured expression of the additional gene(s) is then compared to a reference value of the respective expression level of said genes, and it will thus be possible to conclude that there is an increased risk of death of said subject when the comparison of the expression level of the ADGRE3 gene, particularly at the mRNA level, with a predetermined threshold value, shows that there is a decrease in the expression level, or under-expression, and when the comparison of the level of mRNA transcripts of the additional genes below to a reference value for the respective expression level thereof shows that there is:
an increase in the expression level, or over-expression, of C3AR1, and/or
an increase in the expression level, or over-expression, of CD177, and/or
an increase in the expression level, or over-expression, of OAS2, and/or
a decrease in the expression level, or under-expression, of CIITA, and/or
an increase in the expression level, or over-expression, of IL-10, and/or
an increase in the expression level, or over-expression, of IL1R2, and/or
a decrease in the expression level, or under-expression, of CD74, and/or
an increase in the expression level, or over-expression, of TDRD9.

According to a preferred embodiment, the biological sample is a blood sample, preferably a total blood sample.

According to another preferred embodiment, the expression of ADGRE3, and optionally of the additional genes, is measured at the messenger RNA (mRNA) level.

According to a preferred embodiment, the expression is measured using a molecular detection method, for instance amplification, sequencing or hybridization. The expression is preferably measured by amplification using RT-PCR, particularly RT-qPCR.

According to a preferred embodiment, the measured expression of ADGRE3, and optionally of the additional genes, is normalized in relation to the expression of one or more housekeeping genes. Preferably, the expression is normalized in relation to housekeeping genes selected from DECR1, HPRT1, PPIB, GAPDH, ACTB and combinations thereof.

Another subject of the invention relates to a kit for the in vitro or ex vivo measurement of the expression of ADGRE3 in a biological sample, comprising means for determining the ADGRE3 expression level in said sample. The determination means are preferably selected from amplification primers or probes. Advantageously, the kit comprises a positive control sample calibrated to contain the quantity of ADGRE3 that corresponds to the quantity or concentration representative of the expression level, measured in a pool of samples from subjects who are not infected with a respiratory virus or subjects who are infected with a respiratory virus but are known to have survived, and/or a negative control sample calibrated to contain the quantity of ADGRE3 that corresponds to the average quantity measured in a pool of samples from subjects who did not survive following infection with a respiratory virus.

The kit according to the invention may also comprise means for determining the expression level of at least one other additional gene selected from C3AR1, CD177, OAS2, CIITA, IL-10, IL1R2, CD74, TDRD9 and combinations thereof, preferably selected from C3AR1, CD177, OAS2, CIITA, IL-10, IL1R2 and combinations thereof, and more preferably selected from C3AR1, OAS2, CIITA, IL-10 and combinations thereof. Preferably, said determination means are preferably selected from amplification primers or probes.

Finally, another subject of the invention relates to the use of the kit according to the invention for determining the risk of death, preferably the risk of death in the 28 days following admission to a healthcare facility or in the 37 days post-infection, of a subject infected with a respiratory virus such as SARS-COV-2 or a variant thereof.

### DESCRIPTION OF THE EMBODIMENTS

Certain terms and expressions used in the context of the invention are detailed hereinbelow.

A first subject of the invention relates to an in vitro or ex vivo method for determining the risk of death of a subject infected with a respiratory virus, said method comprising the following steps:
a) measuring the expression level of the ADGRE3 gene in a biological sample from said subject,
b) comparing the expression level of the ADGRE3 gene measured in step a), or a value derived from this quantity, with a predetermined reference value.

Entirely surprisingly, it has been observed that measuring the expression of the ADGRE3 gene made it possible to determine or identify a risk of death of a subject infected with a respiratory virus. Thus, in the context of the increasing importance of personalized medicine, subjects having an increased risk of death could benefit from personalized treatment. Moreover, considering the global prevalence of respiratory viruses such as SARS-COV-2 and associated diseases, particularly the severe forms thereof, it is essential to provide as full an arsenal as possible to be able to quickly and effectively determine the risk of death of patients infected with these viruses.

The method which is a subject of the invention has the advantage of being able to easily assess the increased risk of death of a subject, for example a patient admitted to a healthcare facility such as the resuscitation unit or emergency department, using an easily measurable biomarker, the measurement of which can be carried out directly in the healthcare facility where the subject has been admitted or in a nearby laboratory. Moreover, the measurement of the biomarker ADGRE3, like that of the additional biomarkers of the invention, is entirely suited to being performed by automated analysis devices or “rapid” tests.

“Biomarker” or “marker” means a biological characteristic which can be objectively measured and which is indicative of normal or pathological biological processes or of a pharmacological response to a therapeutic intervention. For the purposes of the present description, the biomarkers are genes and the expression level thereof can be detected in particular at the transcript level, particularly mRNA transcripts.

The ADGRE3 gene (also known under the name EMR3) is located on chromosome 19, from position 14 619 117 to position 14 674 844 (GRCh38/hg38), i.e. 55 728 bp. This gene encodes a member of the class B seven-pass-transmembrane domain receptor family (7TM), which are expressed mainly by cells of the immune system. The members of this family are characterized by an extended extracellular region with a variable number of epidermal growth factor (EGF)-like N-terminal domains, coupled to a 7TM domain via a mucin-like spacer domain. This gene is closely linked to the gene encoding EGF-like module-containing mucin-like hormone receptor 2, on chromosome 19. Alternative splicing of ADGRE3 gives rise to multiple transcription variants encoding different isoforms.

The nucleotide sequence of the ADGRE3 gene is known to those skilled in the art and can be accessed through the NCBI under reference NC_000019.10 (assembly GRCh38.p14).

The expression “risk of death of a subject” refers to the risk of the subject dying in the days following infection with a respiratory virus or in the days following admission to a healthcare facility following infection. In particular, reference is made to an increased risk of death when the subject has a statistically significant risk of death in the days following infection, generally compared to infected subjects who are known to have survived, or uninfected subjects.

Advantageously, the method according to the invention makes it possible to determine an increased risk of death in a subject in the 37 days following the date on which infection is confirmed, also referred to as days post-infection, for example using a test for detecting a respiratory virus.

Thus, the risk of death corresponds to the risk of death of the subject in the 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 days following infection, also referred to as days post-infection. Preferably, the risk of death corresponds to the risk of death of the subject in the 16 days, 23 days, 30 days, 31 days, 32 days, 33 days, 34 days, 35 days, 36 days or 37 days post-infection of the subject. More preferably, the risk of death corresponds to the risk of death in the 37 days post-infection.

According to a particular embodiment, the risk of death may also correspond to the number of days following admission to a healthcare facility, also referred to as days post-admission, it being understood that, at the time of admission, subjects have generally already been infected with a respiratory virus for a number of days. Thus, according to this embodiment, the risk of death corresponds to the risk of death of the subject in the 2, 3, 4, 5, 6, 7, 8, 9, 10, 11, 12, 13, 15, 15, 16, 17, 18, 19, 20, 21, 22, 23, 24, 25, 26, 27 or 28 days post-admission of the subject to a healthcare facility. Preferably, the risk of death corresponds to the risk of death of the subject in the 7 days, 14 days, 21 days, 22 days, 23 days, 24 days, 25 days, 26 days, 27 days or 28 days post-admission of the subject. More preferably, the risk of death corresponds to the risk of death in the 28 days post-admission.

For the purposes of the present description, “healthcare facility” denotes a hospital or clinic, preferably the emergency department, resuscitation department, intensive care unit (ICU) or ongoing care unit, or else a medicalized establishment for the elderly, such as an assisted living facility.

The term “subject” denotes a human, and the subject is preferably a patient. The patient is a person who has been in contact with a healthcare professional, particularly a doctor, or a medical structure or healthcare facility.

According to a particular embodiment, the subject is a patient within a healthcare facility, preferably within a hospital, more preferably within the emergency department, resuscitation department, intensive care unit (ICU) or ongoing care unit, most particularly a patient in the ICU.

“Respiratory virus” means a virus which infects the respiratory tract and/or the lungs. Such viruses are conventionally found in samples taken from the nose, throat and/or mouth of a subject, particularly in nasal samples or nasopharyngeal samples (which require a sample to be taken from more deeply within the nose), oropharyngeal samples (which require a sample to be taken from more deeply in the throat), or saliva.

The expression “subject infected with a respiratory virus” means a subject for whom the test for detecting the presence of a respiratory virus has returned a positive result. These subjects, particularly those who develop more severe forms of the disease, are subjects for whom it is even more relevant to implement the method according to the invention, measuring the ADGRE3 expression level.

Conversely, a subject uninfected with a respiratory virus is a subject for whom the rest for detecting the presence of a respiratory virus is negative.

As examples of respiratory viruses, mention may be made of seasonal coronaviruses, the SARS-COV-2 virus (“Severe Acute Respiratory Syndrome Coronavirus-2”), regardless of the variants thereof, the flu virus, respiratory syncytial virus (RSV), rhinoviruses, metapneumoviruses, parainfluenza viruses and adenoviruses. To date, the known variants of the SARS-COV-2 are in particular the variants thereof referred to as British, Brazilian, South African, American, Indian or Omicron. Knowledge of these variants and their names is evolving, and the invention is applicable to all of them (https://www.who.int/en/activities/tracking-SARS-COV-2-variants/).

The “British variant” of SARS-COV-2 was discovered on 20 Sep. 2020 in Kent (South-East England). The United Kingdom informed the World Health Organization (WHO) of the circulation of this variant on the 14 December. Initially referred to as VUI 202012/01 (for Variant Under Investigation, year 2020, month 12, variant 01), it was soon renamed, on 18 Dec. 2020, as VOC 202012/01 (for Variant Of Concern), then as Alpha variant. It belongs to the B.1.1.7 lineage in the phylogenetic tree, and comprises the deletion 69-70, also referred to as ΔH69/V70. The Brazilian variant P.1 (Gamma variant) is a descendent of the B.1.1.28 lineage. This Brazilian variant P.1 contains numerous mutations, in particular the mutations E484K, K417T and N501Y. The Japanese variant, which is derived from a lineage present in Brazil (B.1.1.28), contains a very high number of genetic alterations. It comprises twelve amino acid mutations in the spike protein, particularly the mutations N501Y, E484K and K417T. The South African variant (Beta variant) referred to as 501Y.V2 and belonging to the lineage B.1.351, also contains various mutations including three, K417N, E484K and N501Y, which are located in the RBD domain, the receptor binding domain, of the spike protein. The Indian variant, referred to as the Delta variant, belongs to the lineage B.1.617.2 and contains the mutations S417N and S484K. Finally, there is also the Omicron variant, identified in November 2021 and belonging to the lineage B.1.1.529.

According to a particular embodiment, the respiratory virus is a coronavirus, in particular SARS-COV-2 or a variant thereof, for instance the Alpha, Beta, Gamma, Delta or Omicron variants.

The expressions “test for detecting a respiratory virus”, “test for diagnosing a respiratory virus” or “test for detecting the presence of an infection with a respiratory virus” are synonymous and refer to any test known to those skilled in the art making it possible to draw such a conclusion, particularly tests for detecting the DNA or RNA of said respiratory virus, such as PCR tests, antigen tests or self-tests.
“Biological sample” refers here to any sample originating from a subject, which may be of different natures, such as blood or derivatives thereof, sputum, urine, stools, skin, cerebrospinal fluid, bronchoalveolar lavage fluid, abdominal cavity puncture fluid, saliva, gastric secretions, sperm, seminal fluid, tears, spinal cord, trigeminal nerve ganglion, adipose tissue, lymphoid tissue, placental tissue, gastrointestinal tract tissue, genital tract tissue, or central nervous system tissue.

In particular, the biological sample may be a biological fluid, such as a blood sample or a blood-derived sample, which may particularly be chosen from total blood (as collected from a vein, i.e. containing white and red blood cells, platelets and plasma), plasma, serum, and any types of cells extracted from the blood, for instance peripheral blood mononuclear cells (PBMCs, containing B lymphocytes, T lymphocytes, NK cells, dendritic cells and monocytes), subsets of B cells, purified monocytes, or neutrophils.

According to a preferred embodiment, the biological sample implemented in the method according to the invention is a blood sample, preferably a total blood sample.

For the purposes of the present description, the biological sample from a subject, namely from a subject for whom the risk of death is to be determined, corresponds to the biological sample to be tested, or test sample, as opposed to the reference sample used for comparison.

For the purposes of the present invention, the expression “reference value” or “predetermined reference value” is synonymous with the expressions “control value” or “threshold value” and serves as a point of comparison for determining whether the expression level of a target gene is decreased or increased.

According to the method which is a subject of the invention, the risk of death of a subject infected with a respiratory virus is particularly determined by implementing a step of measuring the expression level of the ADGRE3 gene.

Measuring the expression level of a gene is well known to those skilled in the art and consists particularly in quantifying at least one expression product of the gene. For the purposes of the present invention, the expression product of a gene may be any biological molecule resulting from the expression of said gene. More particularly, the expression product of the gene may be a transcript.

“Transcript” means RNA, and in particular messenger RNA (mRNA) resulting from the transcription of the gene. More specifically, the transcripts are RNAs produced by the transcription of a gene followed by post-transcriptional modifications of the pre-RNA forms.

According to a preferred embodiment, the measurement of the expression level of ADGRE3, and optionally that of one or more additional genes as listed below, is carried out at the RNA level, in particular at the messenger RNA (mRNA) level, in a biological sample from a subject infected with a respiratory virus. According to this embodiment, the measurement of the expression level of the ADGRE3 gene therefore relates to determining the level of mRNA of said gene.

ADGRE3 gene transcripts are known to those skilled in the art. As examples, mention is made of the transcripts having the following references in the NCBI database: XM_011528374.3 (2298nt), NM_001289159.2 (1971nt), NM_001289158.2 (2193 nt), XM_047439546.1 (2151nt) and NM_032571.5 (2349nt).

As mentioned previously, measuring the expression level of a gene is well known to those skilled in the art. In the case of a transcript, for instance mRNA, the measurement may be performed by a direct method, by any process known to those skilled in the art which makes it possible to determine the presence of said transcript in the biological sample, or by indirect detection of the transcript after conversion of the latter into DNA, or after amplification of said transcript or after amplification of the DNA obtained after conversion of said transcript into DNA. Numerous methods exist for the detection of nucleic acids and are well known to those skilled in the art (see for example Kricka et al., Clinical Chemistry, 1999, no. 45 (4), p. 453-458; Relier G. H. et al., DNA Probes, 2nd Ed., Stockton Press, 1993, sections 5 and 6, p. 173-249).

By way of example, expression of the gene can be determined as follows:
(1) extraction of total RNAs from a blood sample or PBMCs and performing a step of reverse transcription in order to obtain the various complementary DNAs (or cDNA) to the various messenger RNAs initially present in the sample or the PBMCs,
(2) specific amplification of the cDNAs. In this case, the specific reagent used comprises at least one specific amplification primer for the gene. This step can be carried out by a PCR-type amplification reaction or by any other suitable amplification technique,
(3) determining the expression of the gene by quantifying the cDNAs.

The expression of the genes may particularly be measured by Reverse Transcription-Polymerase Chain Reaction or RT-PCR, preferably by quantitative RT-PCR or RT-qPCR (for example using the FilmArray® technology or the Biomark™ platform from Fluidigm), by sequencing (preferably by high-throughput sequencing) or by hybridization techniques (for example with hybridization microarrays or by techniques of the NanoString® nCounter® type). All of these methods are also well known to those skilled in the art, and it is not necessary to describe them in detail here.

According to a particular embodiment, the ADGRE3 expression is measured using a molecular detection method, particularly by RT-PCR, sequencing or hybridization. The expression is preferably measured by RT-PCR, and particularly by RT-qPCR.

According to a particular embodiment, the expression level of the ADGRE3 gene is measured by quantitative RT-qPCR detection of the mRNA transcripts of said gene. Those skilled in the art are entirely able to determine the primers required for the amplification of at least one transcript of the ADGRE3 gene, and optionally of the additional genes, in order to determine the expression level(s).

The measurement of the expression level makes it possible to determine the quantity of one or more ADGRE3 transcripts present in the biological sample or also to give a value derived therefrom.

Thus, according to a particular embodiment, the expression level of the ADGRE3 gene is a value derived from the quantity of transcripts, particularly mRNA, thereof. By way of example, a value derived from the quantity of transcripts may for example be the absolute concentration, calculated by virtue of a calibration curve obtained from successive dilutions of a solution of amplicons having a given concentration. The derived value may also correspond to the value of the normalized and calibrated quantity, such as the CNRQ (Calibrated Normalized Relative Quantity, (Hellemans et al (2007), Genome biology 8 (2):R19), which integrates the values of a reference sample (or of a calibrator) and of one or more housekeeping genes (also referred to as reference genes). By way of example of housekeeping genes, mentioned may be made of the genes DECR1, HPRT1, PPIB, RPLP0, PPIA, GLYR1, RANBP3, 18S, B2M, TBP, GAPDH and ACTB.

According to a particular embodiment, the expression of ADGRE3 is normalized in relation to the expression of one or more housekeeping genes (or reference genes) according to methods known to those skilled in the art. Thus, the expression is normalized using one or more of the following housekeeping genes: DECR1 (chromosomal location: chr8, 90001352-90053633), HPRT1 (chromosomal location: chrX, 134452842-134520513) and PPIB (chromosomal location: chr15:64155812-64163205), RPLP0 (chromosomal location: chr12, 120196699-120201111), PPIA (chromosomal location: chr7, 44795960-44803117), GLYR1 (chromosomal location: chr16, 4803203-4847288), RANBP3 (chromosomal location: chr19, 5916139-5978140), B2M (chromosomal location: chr15, 44711492-44718145), TBP (chromosomal location: chr6, 170554369-170572859), GAPDH (chromosomal location: chr12, 6534517-6538371) and ACTB (chromosomal location: chr14, 5527148-5530601). The chromosomal locations are given according to GRCh38/hg38. Preferably, the expression is normalized using one or more housekeeping genes selected from: DECR1, HPRT1, PPIB, GAPDH, ACTB and combinations thereof, more preferably selected from DECR1, HPRT1, PPIB and combinations thereof.

In such a case, the reference level used is also normalized beforehand, in the same way. The normalization, whether for the reference level or for the level of transcripts of the biological sample to be tested, is carried out before the comparison, particularly before the calculation of a relationship between the level of transcripts of said sample to be tested and the reference level. When a threshold value different from a reference level is used to draw a conclusion, this normalization may be taken into account for the choice of the threshold value.

In the event that the level of ADGRE3 transcripts is normalized relative to the level of transcripts of one or more housekeeping genes, this of course implies that the method according to the invention includes determining the level of transcripts of the housekeeping gene(s) used for the normalization.

Generally speaking, in the method according to the invention, regardless of the embodiments thereof, the ADGRE3 expression level, preferably the normalized expression, in the biological sample from the subject is compared to a reference value or to the expression of the same gene, preferably the normalized expression, obtained in a reference biological sample.

According to a particular embodiment, the expression level of the ADGRE3 gene is determined from a biological sample from a subject, and it can be concluded that there is an increased risk of death of said subject when the comparison of the expression level of said ADGRE3 gene with a predetermined reference value shows that there is a significant difference to said reference value corresponding to said gene. More specifically, said difference corresponds to a level of transcripts, particularly mRNA, for the biological sample to be tested which is less than that corresponding to the reference value. In other words, it can be concluded that there is an increased risk of death of said subject when under-expression of the ADGRE3 gene is demonstrated.

“Under-expression” means a significant decrease in the expression level compared to a reference value. Those skilled in the art are able to determine the statistical test to be used to determine this reference value with which the ADGRE3 expression level is to be compared. The exemplary embodiments present one of the possible methods.

Advantageously, according to a first variant, said reference value corresponds to a reference expression level of the ADGRE3 gene which is the level of transcripts of said gene obtained from a biological sample from a subject who is not infected with any respiratory virus. According to this variant, the reference value may also correspond to the average expression level of ADGRE3 transcripts obtained from biological samples originating from a population of subjects who are not infected with any respiratory virus.

According to a second variant, the reference value corresponds to a reference expression level of the ADGRE3 gene which is the level of transcripts of said gene obtained from a biological sample from a subject who is infected with a respiratory virus but who is known to have survived after infection, particularly in the 37 days following infection. According to this variant, the reference value can also correspond to the average expression level of ADGRE3 transcripts obtained from biological samples originating from a population of subjects who are infected with a respiratory virus but who are known to have survived after infection, particularly in the 37 days following infection.

Preferably, the difference between the level of transcripts of the ADGRE3 gene determined in the test biological sample and the reference level of said gene (reference value) corresponds to the fact that the level of transcripts of said gene determined in the test sample is significantly decreased, in particular by at least 10%, at least 20%, at least 30%, at least 40%, at least 50%, at least 60%, at least 70%, at least 80% or at least 90% relative to the reference expression level, or reference value, of said ADGRE3 gene.

The decrease considered to be relevant for drawing a conclusion will depend on the reference value in question, and particularly on the reference level used as a reference value, and can be adapted by those skilled in the art. In particular, if the reference value corresponds to the reference level of the ADGRE3 gene which is the level of transcripts of said gene in a subject who is not infected with any respiratory virus, in a population of subjects who are not infected with any respiratory virus, or in a subject or population of subjects who are infected with a respiratory virus but who survived in the 37 days following infection, it may be sufficient for the level of ADGRE3 transcripts determined in the test biological sample to simply be less than said reference threshold value.

According to the invention, the method makes it possible to conclude that there is an increased risk of death of the subject when under-expression of ADGRE3 is demonstrated in the biological sample to be tested.

The method according to the invention may thus comprise the following steps:
determining the expression level of the ADGRE3 gene by measuring the quantity of at least one ADGRE3 transcript in a biological sample from said subject,
comparing the quantity of said transcript determined for said biological sample, or a value derived from this quantity, to a reference value predetermined from a reference sample, and
drawing a conclusion regarding the presence of an increased risk of death when the result of the comparison shows a decrease in the expression of the ADGRE3 gene.

According to this embodiment, the reference sample may for example be a sample originating from a subject or a mixture of samples from a plurality of subjects, said subjects not being infected with a respiratory virus. The reference value may particularly correspond to an average value of the ADGRE3 expression level measured from a plurality of samples each originating from different subjects who are not infected with a respiratory virus or who are infected with a respiratory virus but who are known to have survived, particularly in the 28 days following admission to a healthcare facility or in the 37 days post-infection.

According to a preferred variant of this embodiment, the reference sample is of the same nature as the biological sample to be tested, or at least of a compatible nature to form a reference for determining the ADGRE3 expression level.

A “comparison” or verification of a “difference” between two values or levels of values can be carried out by any known technique. The comparison or verification of a “difference” may involve calculating a relationship or a difference.

In the context of the invention, drawing a conclusion regarding the risk of death of the subject from which the test biological sample originates may also be carried out by any automated technique performed by a computer or assisted by a computer.

The method as described previously, in all embodiments thereof, may also comprise, aside from the step of measuring the expression of the ADGRE3 gene, a step of measuring the expression of one or more additional genes.

Thus, according to this particular embodiment, the additional gene(s) are selected from C3AR1, CD177, OAS2, CIITA, IL-10, IL1R2, CD74, TDRD9 and combinations thereof. According to this embodiment, the method also comprises a step of comparing the expression level of the additional gene(s) to a reference value of the respective expression level of said additional genes. The reference values of the additional genes are as defined previously with ADGRE3.

The chromosomal locations of the additional genes are given in table 1 below:

TABLE 1

| | Chromosomal | |
|---|---|---|
| | location | Ensembl & NCBI |
| Gene (name) | (GRCh38/hg38) | accession number |
| C3AR1 | Chromosome 12: | ENSG00000171860 |
| (complement C3a | 8,056,844- | NC_000012.12 |
| receptor 1) | 8,066,359 | |
| CD177 (CD177 | Chromosome 19: | ENSG00000204936 |
| molecule) | 43,353,686- | NC_000019.10 |
| | 43,363,172 | |
| OAS2 (2′-5′- | Chromosome 12: | ENSG00000111335 |
| oligoadenylate | 112,978,395- | NC_000012.12 |
| synthetase 2) | 113,011,723 | |
| CIITA (class II major | Chromosome 16: | ENSG00000179583 |
| histocompatibility | 10,866,222- | NC_000016.10 |
| complex | 10,943,021 | |
| transactivator) | | |
| IL-10 (Interleukin | Chromosome 1: | ENSG00000136634 |
| 10) | 206,767,602- | NC_000001.11 |
| | 206,774,541 | |
| IL1R2 (interleukin 1 | Chromosome 2: | ENSG00000115590 |
| receptor type 2) | 101,991,960- | NC_000002.12 |
| | 102,028,544 | |
| CD74 (CD74 | Chromosome 5: | ENSG00000019582 |
| molecule) | 150,401,639- | NC_000005.10 |
| | 150,412,910 | |
| TDRD9 (tudor | Chromosome 14: | ENSG00000156414 |
| domain containing | 103,928,456- | NC_000014.9 |
| 9) | 104,052,667 | |

Thus, it can be concluded that there is an increased risk of death of said patient when the comparison of the expression level of the ADGRE3 gene, particularly of the mRNA transcripts of said gene, to a predetermined reference value shows that there is a decrease in the expression level, or under-expression, and when the comparison of the expression level of the additional genes below, particularly of the mRNA transcripts of said genes, to a reference value for the respective expression level thereof, shows that there is:
an increase in the expression level, or over-expression, of C3AR1, and/or
an increase in the expression level, or over-expression, of CD177, and/or
an increase in the expression level, or over-expression, of OAS2, and/or
a decrease in the expression level, or under-expression, of CIITA, and/or
an increase in the expression level, or over-expression, of IL-10, and/or
an increase in the expression level, or over-expression, of IL1R2, and/or
a decrease in the expression level, or under-expression, of CD74, and/or
an increase in the expression level, or over-expression, of TDRD9.

According to a variant of this embodiment, the method comprises, regarding the measurement of gene expression, aside from the step of measuring the expression of the ADGRE3 gene, only a step of measuring the expression of one or more additional genes selected from C3AR1, CD177, OAS2, CIITA, IL-10, IL1R2, and combinations thereof, preferably from C3AR1, CD177, CIITA, IL-10, IL1R2 and combinations thereof.

According to another variant of this particular embodiment, the method comprises, regarding the measurement of gene expression, aside from the step of measuring the expression of the ADGRE3 gene, only a step of measuring the expression of the additional gene IL-10.

According to another variant of this embodiment, the method comprises, regarding the measurement of gene expression, aside from the step of measuring the expression of the ADGRE3 gene, only a step of measuring the expression of the additional gene IL-10 and a step of measuring the expression of at least one other additional gene selected from C3AR1, CD177, OAS2, CIITA, IL1R2, CD74, TDRD9 and combinations thereof, preferably selected from C3AR1, CD177, OAS2, CIITA and combinations thereof, and more preferably selected from C3AR1, CIITA and combinations thereof.

According to another variant of this particular embodiment, the method comprises, regarding the measurement of gene expression, aside from the step of measuring the expression of the ADGRE3 gene, only a step of measuring the expression of the additional gene CIITA.

According to another variant of this particular embodiment, the method comprises, regarding the measurement of gene expression, aside from the step of measuring the expression of the ADGRE3 gene, only a step of measuring the expression of the additional gene CIITA and a step of measuring the expression of at least one other additional gene selected from C3AR1, CD177, OAS2, IL-10, IL1R2, CD74, TDRD9 and combinations thereof, preferably selected from C3AR1, CD177, OAS2, IL-10 and combinations thereof, and more preferably selected from C3AR1, IL-10 and combinations thereof.

According to another variant of this particular embodiment, the method comprises, regarding the measurement of gene expression, aside from the step of measuring the expression of the ADGRE3 gene, only a step of measuring the expression of the additional gene CD74.

According to another variant of this particular embodiment, the method comprises, regarding the measurement of gene expression, aside from the step of measuring the expression of the ADGRE3 gene, only a step of measuring the expression of the additional gene CD74 and a step of measuring the expression of at least one other additional gene selected from C3AR1, CD177, OAS2, IL-10, IL1R2, CIITA, TDRD9 and combinations thereof, preferably from C3AR1, CD177, OAS2, IL-10, IL1R2, CIITA and combinations thereof, and more preferably from C3AR1, IL-10, CIITA and combinations thereof.

According to another variant of this embodiment, the method comprises, regarding the measurement of gene expression, aside from the step of measuring the expression of the ADGRE3 gene, only a step of measuring the expression of the additional gene OAS2.

According to another variant of this particular embodiment, the method comprises, regarding the measurement of gene expression, aside from the step of measuring the expression of the ADGRE3 gene, only a step of measuring the expression of the additional gene OAS2 and a step of measuring the expression of at least one other additional gene selected from C3AR1, CD177, CD74, IL-10, IL1R2, CIITA, TDRD9 and combinations thereof, preferably selected from C3AR1, CD177, IL-10, IL1R2, CIITA and combinations thereof, and more preferably selected from C3AR1, IL-10, CIITA and combinations thereof.

According to another variant of this embodiment, the method comprises, regarding the measurement of gene expression, aside from the step of measuring the expression of the ADGRE3 gene, only a step of measuring the expression of the additional gene C3AR1.

According to another variant of this particular embodiment, the method comprises, regarding the measurement of gene expression, aside from the step of measuring the expression of the ADGRE3 gene, only a step of measuring the expression of the additional gene C3AR1 and a step of measuring the expression of at least one other additional gene selected from OAS2, CD177, CD74, IL-10, IL1R2, CIITA, TDRD9 and combinations thereof, preferably selected from OAS2, CD177, IL-10, IL1R2, CIITA and combinations thereof, and more preferably selected from IL-10, CIITA and combinations thereof.

According to another variant of this embodiment, the method comprises, regarding the measurement of gene expression, aside from the step of measuring the expression of the ADGRE3 gene, only a step of measuring the expression of the additional gene IL1R2.

According to another variant of this particular embodiment, the method comprises, regarding the measurement of gene expression, aside from the step of measuring the expression of the ADGRE3 gene, only a step of measuring the expression of the additional gene IL1R2 and a step of measuring the expression of at least one other additional gene selected from C3AR1, CD177, CD74, IL-10, OAS2, CIITA, TDRD9 and combinations thereof, preferably selected from C3AR1, CD177, IL-10, OAS2, CIITA and combinations thereof, and more preferably selected from C3AR1, IL-10, CIITA and combinations thereof.

According to another variant of this embodiment, the method comprises, regarding the measurement of gene expression, aside from the step of measuring the expression of the ADGRE3 gene, only a step of measuring the expression of the additional gene TDRD9.

According to another variant of this embodiment, the method comprises, regarding the measurement of gene expression, aside from the step of measuring the expression of the ADGRE3 gene, only a step of measuring the expression of the additional gene TDRD9 and a step of measuring the expression of at least one other additional gene selected from C3AR1, CD177, OAS2, IL-10, IL1R2, CIITA and combinations thereof, preferably from C3AR1, CD177, OAS2, IL-10, IL1R2, CIITA and combinations thereof, and more preferably from C3AR1, OAS2, IL-10, CIITA and combinations thereof.

According to another variant of this embodiment, the method comprises, aside from the step of measuring the expression of the ADGRE3 gene, only a step of measuring the expression of the following 8 additional genes: C3AR1, CD177, OAS2, CIITA, IL-10, IL1R2, CD74 and TDRD9.

According to a particularly preferred variant, the method according to the invention comprises, jointly with the measurement of the expression of the ADGRE3 gene, the measurement of the expression of the additional genes presented in table 2:

TABLE 2

| | |
|---|---|
| Combinations | C3AR1, CIITA |
| of 2 additional | C3AR1, IL-10 |
| genes | C3AR1, OAS2 |
| | CIITA, IL-10 |
| | CIITA, OAS2 |
| | IL-10, OAS2 |
| Combinations | C3AR1, CIITA, IL-10 |
| of 3 additional | C3AR1, CIITA, OAS2 |
| genes | C3AR1, IL-10, OAS2 |
| | CIITA, IL-10, OAS2 |
| Combinations | C3AR1, CIITA, IL-10, OAS2 |
| of 4 additional | C3AR1, CD74, IL-10, TDRD9 |
| genes | |

For all the variant embodiments described previously, the method comprises a step of concluding regarding an increased risk of death on the basis of the measurement of the expression levels of said genes and of the comparison of the levels measured in this way (increase or decrease) to reference values determined as described previously.

Another subject of the invention relates to a kit for the in vitro or ex vivo measurement of the expression level of the ADGRE3 gene in a biological sample from a subject, said kit comprising at least one means for determining the ADGRE3 expression level in said biological sample.

The means for determining the expression level of the ADGRE3 gene are known to those skilled in the art and can be specific tools or reagents that make it possible to measure said expression in a biological sample. They may for example be primers or probes.

Of course, the specific or preferred embodiments described in association with the methods according to the invention apply to the kits and uses which are also subject of the invention.

“Primer” or “amplification primer” means a nucleotide fragment which may consist of 5 to 100 nucleotides, preferably 15 to 30 nucleotides, and having hybridization specificity with a target nucleotide sequence under conditions determined for the initiation of enzymatic polymerization, for example in a reaction for the enzymatic amplification of the target nucleotide sequence. Generally, use is made of “primer pairs” consisting of two primers. When it is desired to amplify several different biomarkers (e.g. from different genes), several different pairs of primers are preferably used, each preferentially having the ability to hybridize specifically with a different biomarker.

Those skilled in the art are thus able, from the gene sequence and particularly from the corresponding transcripts, to determine the primers and probes required for amplification, particularly for the amplification of one or more mRNA transcripts of said gene.

“Probe” or “hybridization probe” means a nucleotide fragment typically consisting of 5 to 100 nucleotides, preferably 15 to 90 nucleotides, even more preferably 15 to 35 nucleotides, having hybridization specificity under conditions determined for forming a hybridization complex with a target nucleotide sequence. The probe also comprises a reporter (such as a fluorophore, an enzyme or any other detection system) which will enable the detection of the target nucleotide sequence. In the present invention, the target nucleotide sequence may be a nucleotide sequence contained in a messenger RNA (mRNA) or a nucleotide sequence contained in a complementary DNA (cDNA) obtained by reverse transcription of said mRNA. When it is desired to target several different biomarkers (e.g. from different genes), several different probes are preferably used, each preferentially having the ability to hybridize specifically with a different biomarker.

“Hybridization” means the process during which, under suitable conditions, two nucleotide fragments, for example a hybridization probe and a target nucleotide fragment, having sufficiently complementary sequences, are able to form a double strand with stable and specific hydrogen bonds. A nucleotide fragment which is “able to hybridize” with a polynucleotide is a fragment which can hybridize with said polynucleotide under hybridization conditions, which can be determined in each case in a known way. The hybridization conditions are determined by stringency, i.e. the strictness of the operating conditions. Hybridization is more specific when it is carried out at higher stringency levels. The stringency is defined particularly on the basis of the base composition of a probe/target duplex, and also by the degree of mismatch between two nucleic acids. The stringency can also be based on the reaction parameters, such as the concentration and type of the ionic species present in the hybridization solution, the nature and the concentration of denaturing agents, and/or the hybridization temperature. The stringency of the conditions under which a hybridization reaction must be carried out will chiefly depend on the hybridization probes used. All this information is well known and the suitable conditions can be determined by those skilled in the art.

In general, depending on the length of the hybridization probes used, the temperature for the hybridization reaction is between approximately 20 and 70° C., in particular between 35 and 65° C. in a saline solution at a concentration of approximately 0.5 to 1 M. A step of detecting the hybridization reaction is subsequently carried out.

According to a particular embodiment, at least one reference value is stored in a computer-readable medium, for example a bar code, and/or can be used in the form of a code which can be executed by a computer configured to compare the level of ADGRE3 transcripts determined by virtue of the determination means, or an item of data obtained from said level of ADGRE3 transcripts, to said reference value.

According to this embodiment, the reference value may correspond to the level of transcripts, preferably at the mRNA level, of ADGRE3 in a subject who is not infected with any respiratory virus or who is infected with a respiratory virus but who survived in the 37 days following infection. The reference value may also correspond to the average level of transcripts, preferably at the mRNA level, of ADGRE3 in a population of subjects who are not infected with any respiratory virus or who are infected with a respiratory virus but who survived in the 37 days following infection.

According to another embodiment, the kit further comprises at least one reference level for the ADGRE3 marker gene, stored in a computer-readable medium, for example a bar code, and/or used in the form of a code which can be executed by a computer configured to compare the level of ADGRE3 transcripts determined by virtue of the determination means, to said reference value, said reference value preferably being the level of ADGRE3 transcripts in a subject who is not infected with a respiratory virus, or a population of such subjects.

According to another embodiment, the kit further comprises a positive control sample, which is a sample calibrated to contain the quantity of ADGRE3 that corresponds to the quantity or concentration representative of the expression level, measured in a pool of samples from subjects who are known not to be infected with a respiratory virus, and/or a negative control sample, which is a sample calibrated to contain the quantity of ADGRE3 that corresponds to the average quantity measured in a pool of samples from subjects who are known not to have survived, particularly in the 37 days, following infection with a respiratory virus.

According to another embodiment, the kit further comprises a positive control sample, which is a sample calibrated to contain the quantity of ADGRE3 that corresponds to the quantity or concentration representative of the expression level, measured in a pool of samples from subjects who are infected with a respiratory virus and who are known to have survived in the 37 days following infection, and/or a negative control sample, which is a sample calibrated to contain the quantity of ADGRE3 that corresponds to the average quantity measured in a pool of samples from subjects who are known not to have survived, particularly in the 37 days, following infection with a respiratory virus.

In all embodiments thereof, the kit may also comprise at least one additional means for determining the expression level of one or more additional genes selected from C3AR1, CD177, OAS2, CIITA, IL-10, IL1R2, CD74, TDRD9 and combinations thereof.

According to this embodiment, the kit may comprise, in the same way as defined previously, a reference value for the expression level of the additional genes and/or of the (positive or negative) control samples of said additional genes. The means for determining the expression level may be specific tools or reagents as defined previously, for example primers or probes.

According to a preferred embodiment, the kit may also comprise, aside from a means for determining the ADGRE3 expression level, at least one other additional means for determining the expression level of one or more additional genes selected from C3AR1, CD177, OAS2, CIITA, IL-10, IL1R2 and combinations thereof, preferably selected from C3AR1, OAS2, CIITA, IL-10 and combinations thereof, from a biological sample from a subject.

According to this embodiment, the kit may also comprise a reference value for the expression level of the additional genes and/or of the (positive or negative) control samples of said additional genes.

Another subject relates to the use of a kit for in vitro or ex vivo measurement, as defined previously, for determining the risk of death of a subject infected with a respiratory virus.

The invention also relates to the in vitro or ex vivo determination methods for determining the risk of death of a subject infected with a respiratory virus, such methods being as defined by the present description and further comprising a step of treating the infection with said respiratory virus.

In particular, treatment can be initiated as soon as a conclusion is drawn regarding the risk of death of said subject infected with a respiratory virus.

The treatment may consist in administering a suitable antiviral drug. As antiviral treatments, mention may particularly be made of Iopinavir®, Ritonavir®, and recombinant interferons, particularly interferon beta, alpha and lambda. Numerous therapeutic treatments for respiratory viruses, particularly those responsible for COVID-19, are currently in the trial phase (Canedo-Marroquín, G. et al., 2020).

According to a particular embodiment, the treatment step consists in administering one or more monoclonal antibodies to said subject. Examples of such antibodies are casirivimab, imdevimab, regdanvimab, tixagevimab or cilgavimab. Preferred treatments may consist of the combination of casirivimab and imdevimab (Ronapreve), or the combination of tixagevimab and cilgavimab.

Another subject relates to a method comprising the following steps:
obtaining a biological sample, preferably a total blood sample, from a subject infected with a respiratory virus, which is SARS-COV-2,
bringing said biological sample into contact with specific reagents for the expression products of the genes ADGRE3, C3AR1, CD177, OAS2, CIITA, IL-10, IL1R2, CD74, TDRD9, preferably ADGRE3, C3AR1, CD177, OAS2, CIITA, IL-10, IL1R2, and more preferably ADGRE3, C3AR1, CIITA, and IL-10,
measuring the expression of said target genes.

The specific reagents for the expression products are selected from amplification primers, hybridization probes or antibodies, and are as defined previously.

According to a particular embodiment, the method may comprise a step of administering a suitable antiviral drug as defined previously, particularly when the expression of the target genes indicate that the subject has an increased risk of death in the 37 days.

Another subject relates to a method for determining whether a patient infected with a respiratory virus has an increased risk of death, comprising the following steps of:
obtaining a biological sample, preferably a total blood sample, from a patient infected with a respiratory virus,
measuring the expression level of the ADGRE3 gene in said biological sample,
comparing the expression level of the ADGRE3 gene to a reference value obtained from patients who are infected with a respiratory virus and who survived,
wherein, if the expression level of the ADGRE3 gene is less than the reference value, it is determined that the patient has an increased risk of death.

According to a particular embodiment, the method also comprises measuring the expression level of one or more additional genes as defined previously, and comparing the measured levels with respective reference values for each of the additional genes, also obtained from patients who are infected with a respiratory virus and who survived. In this case, the increased risk of death is determined

Finally, another subject relates to a method comprising the quantitative measurement, particularly by RT-qPCR, of the mRNA of the ADGRE3 gene, and optionally of one or more additional genes as defined previously, in a biological blood sample from a subject infected with a respiratory virus such as SARS-COV-2.

The present invention is illustrated, non-limitingly, using the following examples.

### EXAMPLES

### Materials and Method

### 1. Description of Patient Cohorts

The RICO cohort (REA-IMMUNO-COVID) is an ongoing prospective observational clinical study. In this auxiliary study, 309 patients were recruited between August 2020 and August 2021 from five intensive care units in affiliated university hospitals (Hospices Civils de Lyon, France).

The patients all had a pulmonary infection due to SARS-COV-2. The results from this cohort have been previously published (F. Venet et al., 2021). In brief, the criteria for inclusion were the following: (1) male or female≥18 years old, (2) hospitalization in intensive care unit (ICU) for SARS-COV-2 respiratory infection, (3) first ICU hospitalization, (4) positive diagnosis of infection with SARS-COV-2 by PCR or by another approved method in at least one respiratory sample, (5) possibility to take blood sample in the first 24 h following admission to ICU (D0), and (6) patient or next of kin having been informed about the terms of the study and not being opposed to participation.

The criteria for exclusion were pregnancy, institutionalized patients and where informed consent could not be obtained.

From this cohort, samples were taken and analyzed on DO. The patients had an average age of 65 years [InterQuartile Range (IQR), 57-72]) and exhibited a disparate distribution of men to women (68/32).

Cohort of healthy volunteers: blood samples from 49 volunteers were obtained independently from the Etablissement Français du Sang (EFS, Lyon, France). The healthy donors were aged 40 years [IQR, 27-54], with a uniform distribution of men to women. All samples were taken in April 2020 and November 2021.

### 2. Transcriptome Analysis

Blood samples were collected in PAXgene® tubes (ref. 762165, PreAnalytix GmbH Hombrechtikon Switzerland) on D0, following the manufacturer's instructions. In brief, the samples were left at room temperature for 2 h with the reagents present in the tubes, before being transferred to −20° C. for at least 24 h, then stored at −80° C.

The samples were tested using a FilmArray® pouch optimized to detect genes involved in host response, particularly mRNA from genes such as ADGRE3, using nested PCR. The pouches are analyzed using the FilmArray® Torch (BioFire, USA) instrument, following the manufacturer's instructions. The expression level results are obtained in an automated manner, in less than one hour, before being compiled for analysis.

The normalized expression values for the markers (in relation to the reference genes DECR1, HPRT1 and PPIB) were calculated and used for analyses.

### 3. Measurements of Immune Markers

The number of CD3+T lymphocytes of the T lymphocytes was determined using an automated volumetric flow cytometer (Aquios C L, Beckman Coulter).

The standardized values (antibodies/cells or Ab/c) of the expression of HLA-DR by monocytes (mHLA-DR) were obtained using a flow cytometer (Navios, Beckman Coulter) with HLA-DR Quantibrite reagents (Becton Dickinson), as described previously (F. Venet et al., 2021).

### 4. Statistical Analyses

The RICO cohort was divided randomly to obtain two datasets which were balanced according to 3 parameters: age, sex and mortality. This gave a first dataset of 216 patients used for machine learning, and an independent dataset of 93 patients used for validating performance. For describing the datasets, the qualitative data was reported in numerical form or as a frequency, and the quantitative data was reported in the form of a median [IQR range].

The clinical characteristics were compared using the Mann-Whitney-Wilcoxon nonparametric test for continuous variables and Fisher's exact test or chi-squared test (where appropriate) for the categorical variables. Significance was set at 5% for the bilateral tests. The statistical analyses were carried out using the R software, version 3.6.2. The data were centered and reduced to perform unsupervised principal component analysis via the FactoMineR package (version 2.4).

The genes significantly associated with mortality in 28 days in a univariate logistic regression model were used to construct multivariate models for predicting survival at 28 days. The trained models are logistic regression models with regularization L1(Lasso), L2(Ridge) and mixed logistic regression (ElasticNet), Partial Least Squares-Discriminant (PLS) analysis, and linear Support Vector Machines (linear SVM) using the CARET package (version 6.0-84).

In order to compensate for the unbalanced distribution of mortality in the datasets, the synthetic minority oversampling technique (SMOTE) was applied to tune hyperparameters (N. V Chawla et al., 2002).

The hyperparameters of the models were chosen due to the low incidence of the outcome of interest within the cohort (AUPRC) among the cross-validation tests (k-fold-5, number of repetitions=10) in the training RICO cohort (B. Ozenne et al., 2015), with sensitivity, specificity, F1 score and the positive predictive values (PPV) being the parameters of interest.

In summary, among the 5 machine learning algorithms evaluated, the hyperparameters selected are as follows: Lasso (α=1, λ=0.031), Ridge (α=0, λ=0.556), Elastic net (α=0.35, λ=0.37), PLS (ncomp=1), and Linear SVM (C=0.367).

AUPRC, and the 95% bootstrap confidence interval thereof, were obtained using PRROC (version 1.3.1) and boot (version 1.3-28) packages. The bootstrap resampling number was defined as N=1000. The importance of the relative variables in the Linear SVM model was calculated using the FIRM method from the vip package (version 0.3.2) (B Greenwell et al., 2018).

The area under the ROC curve (AUROC), the 95% bootstrap confidence interval, and the diagnostic performance (sensitivity, specificity, positive and negative predictive values and F1 score) at the optimal thresholds for the panel of the 9 mRNAs, and the individual parameters, were obtained taking into account the respective Youden indices from the cutpointr software package (version 1.1.1) defined over the training dataset, then applied to the values of the test dataset. The F1 score (harmonic mean of precision and recall) was used as a measure of the precision of the model due to the data imbalance.

### 5. Ethics

The protocol for the RICO study was approved by the ethics committee (Comité de Protection des Personnes Ile de France 1-IRB/IORG number: IORG0009918) under agreement number 2020-A01079-30. This clinical study was registered at ClinicalTrials.gov (NCT04392401).

### Results

### 1. Clinical Characteristics of Patients on Admission to Intensive Care Unit

The characteristics of the patients are presented in table 3 below. In total, 309 patients were hospitalized in 5 hospitals in Lyon (France) between August 2020 and August 2021 and were included in the cohort.

In brief, and as mentioned previously, 70% of the patients were men. The patients were admitted to the intensive care unit at a median of 9 days from the presentation of the first symptoms [IQR, 6-11], and in particular had a median body mass index (BMI) (kg/m3) of 29.1 [IQR: 26.1-33.2]. In terms of disease severity, the patients had a decreased PaO2/FiO2 (mmHg) with a median of 97.5 [IQR: 74.3-146.5], a high SOFA score [median: 2.0; IQR: 1.0-5.0] and a SAPS II score [median: 30.0; IQR: 23.5-39.0]. In addition, 17.2% of the patients required invasive mechanical ventilation on admission.

All patients were receiving corticosteroid treatment before or after admission (6 mg/day of dexamethasone).

Overall, the patients spent a median duration of 18 days [IQR: 11.0-31.8] in hospital, including 8 days [IQR: 4.0-17.0] in intensive care. A third of the cohort developed secondary infections, the majority of which were pneumopathies (87/99), of which 16 were of fungal origin.

Finally, among the 309 patients, 52 (17%) died by the end of the 28th day post-admission.

TABLE 3

| | | | | Test | | |
|---|---|---|---|---|---|---|
| | Training | | | | Did not | |
| | | Did not | | Survived | survive | |
| | Survived at | survive at 28 | | at 28 | at 28 | |
| All patients | 28 days | days | p | days | days | p |
| (n = 309) | (n = 179) | (n = 37) | value | (n = 78) | (n = 15) | value |

Demographics

| | | | | | | | |
|---|---|---|---|---|---|---|---|
| Age - years | 65.0 | 64.0 | 71.0 | <0.001 | 64.5 | 72.0 | <0.001 |
| | [57.0-72.0] | [55.0-70.0] | [69.0-78.0] | | [55.0-70.0] | [68.0-76.0] | |
| Male - | 210 (68.0%) | 119 (66.5%) | 28 (75.7%) | 0.369 | 52 (66.7%) | 11 (73.3%) | 0.767 |
| n (%) | | | | | | | |
| Body mass | 29.1 | 29.1 | 29.6 | 0.674 | 28.7 | 30.1 | 0.335 |
| index (BMI) - | [26.1-33.2] | [25.7-33.1] | [27.33-33.0] | | [26.1-33.4] | [28.0-33.2] | |
| kg/m² | | | | | | | |
| BMI > 30 - | 128 (43.7%) | 75 (43.9%) | 15 (46.9%) | 0.903 | 30 (40.0%) | 8 (53.3%) | 0.504 |
| n (%) | | | | | | | |

Comorbidities

| | | | | | | | |
|---|---|---|---|---|---|---|---|
| Diabetes: none - | 213 (69%) | 131 (73.2%) | 18 (48.7%) | | 55 (70.5%) | 9 (60.0%) | |
| n (%) | | | | | | | |
| Diabetes: with | 15 (4.9%) | 6 (3.4%) | 6 (16.2%) | 0.001 | 3 (3.9%) | 0 (0.0%) | 0.534 |
| deterioration - n | | | | | | | |
| (%) | | | | | | | |
| Diabetes: | 81 (26.2%) | 42 (23.4%) | 13 (35.1%) | | 20 (25.6%) | 6 (40.0%) | |
| without organic | | | | | | | |
| deterioration - n | | | | | | | |
| (%) | | | | | | | |
| Charlson index - | 1.0 | 1.0 | 2.0 | <0.001 | 0.5 | 1.0 | 0.043 |
| points | [0.0-2.0] | [0.0-1.0] | [1.0-4.0] | | [0.0-1.0] | [1.0-2.0] | |

Clinical severity on admission

| | | | | | | | |
|---|---|---|---|---|---|---|---|
| Time between | 9.0 | 9.0 | 7.5 | 0.013 | 9.0 | 6.0 | 0.041 |
| first symptoms | [6.0-11.0] | [7.0-12.0] | [5.0-9.8] | | [6.8-10.3] | [5.5-9.0] | |
| and admission to | | | | | | | |
| ICU - days | | | | | | | |
| SOFA score - | 2.0 | 2.0 | 4.0 | 0.008 | 2.0 | 3.0 | 0.068 |
| points | [1.0-5.0] | [0.0-5.0] | [2.0-6.0] | | [0.3-3.0] | [1.5-7.5] | |
| SAPS II score - | 30.0 | 30.0 | 39.0 | <0.001 | 27.5 | 32.0 | 0.086 |
| points | [23.5-39.0] | [23.0-38.8] | [33.0-47.0] | | [21.0-34.0] | [26.8-41.3] | |
| | 97.5 | 95.0 | 82.0 | 0.376 | 98.0 | 104.5 | 0.844 |
| PaO₂/FiO₂ - | [74.3-146.5] | [77.5-146.0] | [70.5-147.8] | | [89.0-149.0] | [93.8-128.3] | |
| mmHg | | | | | | | |
| pH | 7.45 | 7.46 | 7.44 | 0.591 | 7.46 | 7.47 | 0.769 |
| | [7.42-7.49] | [7.42-7.49] | [7.40-7.49] | | [7.43-7.49] | [7.40-7.49] | |
| Lactate - mmol/L | 1.65 | 1.70 | 1.90 | 0.326 | 1.50 | 1.40 | 0.785 |
| | [1.30-2.00] | [1.37-2.02] | [1.40-2.20] | | [1.30-1.90] | [1.30-1.80] | |

Organ support

| | | | | | | | |
|---|---|---|---|---|---|---|---|
| Invasive | 53 (17.2%) | 29 (16.2%) | 10 (27%) | 0.186 | 9 (11.5%) | 5 (33.3%) | 0.046 |
| mechanical | | | | | | | |
| ventilation on | | | | | | | |
| day 0 - | | | | | | | |
| n (%) | | | | | | | |
| Vasoactive | 35 (11.4%) | 19 (10.7%) | 8 (21.6%) | 0.120 | 6 (7.7%) | 2 (13.3%) | 0.611 |
| drugs - n (%) | | | | | | | |
| Renal | 31 (10.0%) | 13 (7.3%) | 11 (29.7%) | <0.001 | 4 (5.1%) | 3 (20.0%) | 0.080 |
| replacement | | | | | | | |
| therapy - n (%) | | | | | | | |

Follow-up care

| | | | | | | | |
|---|---|---|---|---|---|---|---|
| MV duration - | 14.0 | 17.0 | 12.0 | 0.110 | 22.5 | 12.0 | 0.030 |
| days | [7.0-27.3] | [7.0-34.0] | [7.0-20.0] | | [11.3-30.8] | [6.5-15.5] | |
| Days in | 8.0 | 8.0 | 12.0 | 0.017 | 8.0 | 11.0 | 0.871 |
| Intensive | [4.0-17.0] | [3.0-16.0] | [8.0-19.0] | | [5.0-16.8] | [5.5-15.5] | |
| Care Unit | | | | | | | |
| Days in | 18.0 | 18.0 | 15.0 | 0.024 | 20.5 | 14.0 | 0.014 |
| hospital | [11.0-31.8] | [10.0-36.5] | [9.0-21.0] | | [13.0-34.8] | [7.5-18.5] | |
| Mortality at 28 | 52 (16.8%) | 0 (0%) | 37 (100%) | <0.001 | 0 (0%) | 15 (100%) | <0.001 |
| days - n (%) | | | | | | | |
| Mortality at 90 | 66 (21.9%) | 12 (6.8%) | 37 (100%) | <0.001 | 2 (2.7%) | 15 (100%) | <0.001 |
| days - n (%) | | | | | | | |
| Infections | 99 (33.1%) | 55 (31.6%) | 19 (54.3%) | 0.018 | 15 (20.0%) | 10 (66.7%) | <0.001 |
| acquired in | | | | | | | |
| ICU - n (%) | | | | | | | |
| Pneumopathies | 87/99 (87.9%) | 48/55 (87.3%) | 18/19 (94.7%) | 0.366 | 12/15 (80.0%) | 9/10 (90.0%) | 0.504 |
| acquired in ICU - | | | | | | | |
| n (% IAI) | | | | | | | |

Immunological parameters on admission

| | | | | | | | |
|---|---|---|---|---|---|---|---|
| mHLA-DR - | 8950.0 | 9246.0 | 7377.5 | 0.029 | 8939.0 | 8967.0 | 0.810 |
| AB/C | [6655.5-12173.5] | [6770.0-12827.0] | [4760.8-11413.0] | | [6859.8-11038.8 | [7551.5-11118.0] | |
| CD3 T | 325.0 | 326.0 | 303.0 | 0.056 | 326.0 | 500.0 | 0.583 |
| lymphocytes - | [228.0-505.5] | [236.5-506.0] | [200.0-400.0] | | [218.0-515.0] | [251.0-555.5] | |
| absolute number | | | | | | | |

The medians and interquartile ranges [Q1-Q3] are indicated for continuous variables where the numbers and percentages are presented for categorical variables. The patients suffering from COVID-19 were separated into two groups according to their survival status 28 days after admission. The sequential organ failure (SOFA) and simplified acute physiology II (SAPS II) scores were calculated during the first 24 hours after admission. Acute respiratory distress on admission was based on the Berlin definition. The data were compared using the Mann-Whitney nonparametric test for continuous variables and/or Fisher's exact test for categorical variables.

### 2. Association Between Expression of Biomarkers and Mortality of Patients 28 Days Post-Admission to a Healthcare Facility

Using univariate logistic regression analysis of a dataset of 216 patients, the ADGRE3 gene was identified as being significantly associated with the mortality at 28 days of patients infected with a respiratory virus. In particular, the results show significant under-expression of ADGRE3 in patients who did not survive, compared to patients who did survive. The results are presented in Table 4 below.

TABLE 4

| | OR_{IQR} [95% CI] | IQR | p value |
|---|---|---|---|
| ADGRE3 | 0.66 [0.45-0.93] | 1.17 | 0.021 |
| mHLA-DR | 0.97 [0.67-1.30] | 6246 | 0.856 |
| [antibodies/cells] | | | |
| CD3 T cells | 0.56 [0.31-0.90] | 258.5 | 0.031 |
| [cells/μL] | | | |

216 patients were included in the training set; 179 survived to the 28th day and 37 died. The association between the survival status at 28 days and the ADGRE3 gene or conventional immune parameters was made by implementing univariate logistic regression models. In order to enable comparison between models, the odds ratios calculated for ADGRE3 and each immune parameter were normalized to an increment of the first to third quartile (odd ratios inter quartile range, ORIQR). The p≤0.05 values are highlighted in bold.

Among the cellular parameters, the measurement of mHLA-DR is not significantly associated with the mortality of patients at 28 days, thereby demonstrating the full interest of the ADGRE3 biomarker.

The ADGRE3 biomarker was then used in the 5 training models in combination with one or more additional biomarkers in order to validate the predictive signatures of the risk of death in patients at 28 days post-admission.

The performance results are presented in the tables below:

### Combination of ADGRE3 and IL-10

TABLE 5

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.715 | [0.615-0.816] | 0.362 | [0.233-0.525] | 0.717 | [0.573-0.861] | 0.351 | [0.174-0.626] |
| Ridge | 0.713 | [0.62-0.805] | 0.364 | [0.217-0.518] | 0.736 | [0.611-0.861] | 0.323 | [0.156-0.583] |
| Lasso | 0.714 | [0.614-0.815] | 0.358 | [0.227-0.506] | 0.718 | [0.574-0.862] | 0.343 | [0.148-0.633] |
| PLS | 0.98 | [0.963-0.997] | 0.847 | [0.701-0.973] | 0.629 | [0.484-0.775] | 0.241 | [0.122-0.453] |
| svmLin | 0.713 | [0.621-0.805] | 0.364 | [0.234-0.511] | 0.74 | [0.617-0.864] | 0.327 | [0.174-0.615] |

### Combination of ADGRE3 and CIITA

TABLE 6

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.652 | [0.556-0.748] | 0.32 | [0.198-0.472] | 0.65 | [0.495-0.806] | 0.236 | [0.118-0.433] |
| Ridge | 0.64 | [0.545-0.736] | 0.316 | [0.185-0.476] | 0.654 | [0.5-0.808] | 0.234 | [0.116-0.44] |
| Lasso | 0.64 | [0.544-0.736] | 0.326 | [0.202-0.487] | 0.653 | [0.499-0.807] | 0.233 | [0.122-0.439] |
| PLS | 0.64 | [0.544-0.736] | 0.327 | [0.192-0.475] | 0.652 | [0.498-0.806] | 0.232 | [0.11-0.412] |
| svmLin | 0.637 | [0.541-0.734] | 0.321 | [0.182-0.469] | 0.657 | [0.502-0.813] | 0.24 | [0.121-0.455] |

### Combination of ADGRE3 and C3AR1

TABLE 7

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.651 | [0.555-0.748] | 0.339 | [0.213-0.493] | 0.61 | [0.478-0.743] | 0.186 | [0.103-0.296] |
| Ridge | 0.648 | [0.548-0.747] | 0.339 | [0.208-0.495] | 0.631 | [0.5-0.762] | 0.195 | [0.109-0.319] |
| Lasso | 0.642 | [0.54-0.743] | 0.336 | [0.189-0.486] | 0.626 | [0.495-0.756] | 0.192 | [0.103-0.311] |
| PLS | 0.648 | [0.548-0.747] | 0.339 | [0.191-0.485] | 0.631 | [0.5-0.762] | 0.195 | [0.11-0.309] |
| svmLin | 0.648 | [0.55-0.746] | 0.336 | [0.204-0.484] | 0.628 | [0.496-0.76] | 0.194 | [0.114-0.322] |

### Combination of ADGRE3 and OAS2

TABLE 8

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.645 | [0.528- | 0.377 | [0.242- | 0.689 | [0.545- | 0.259 | [0.134- |
| | | 0.762] | | 0.54] | | 0.832] | | 0.497] |
| Ridge | 0.625 | [0.508- | 0.354 | [0.212- | 0.669 | [0.531- | 0.233 | [0.129- |
| | | 0.741] | | 0.503] | | 0.808] | | 0.423] |
| Lasso | 0.616 | [0.5- | 0.34 | [0.206- | 0.669 | [0.535- | 0.229 | [0.122- |
| | | 0.732] | | 0.474] | | 0.803] | | 0.45] |
| PLS | 0.625 | [0.508- | 0.355 | [0.217- | 0.67 | [0.531- | 0.233 | [0.122- |
| | | 0.742] | | 0.509] | | 0.809] | | 0.455] |
| svmLin | 0.655 | [0.538- | 0.367 | [0.214- | 0.7 | [0.553- | 0.27 | [0.134- |
| | | 0.773] | | 0.512] | | 0.847] | | 0.504] |

### Combination of ADGRE3, IL-10 and CD74

TABLE 9

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.715 | [0.615- | 0.362 | [0.233- | 0.717 | [0.573- | 0.351 | [0.178- |
| | | 0.816] | | 0.511] | | 0.861] | | 0.673] |
| Ridge | 0.723 | [0.626- | 0.393 | [0.248- | 0.759 | [0.629- | 0.364 | [0.177- |
| | | 0.82] | | 0.561] | | 0.889] | | 0.64] |
| Lasso | 0.716 | [0.615- | 0.362 | [0.236- | 0.718 | [0.574- | 0.354 | [0.174- |
| | | 0.816] | | 0.527] | | 0.862] | | 0.649] |
| PLS | 0.981 | [0.966- | 0.879 | [0.767- | 0.726 | [0.586- | 0.325 | [0.151- |
| | | 0.996] | | 0.978] | | 0.867] | | 0.595] |
| svmLin | 0.717 | [0.619- | 0.387 | [0.246- | 0.762 | [0.628- | 0.416 | [0.2- |
| | | 0.814] | | 0.547] | | 0.897] | | 0.668] |

### Combination of ADGRE3, C3AR1 and CIITA

TABLE 10

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.674 | [0.582- | 0.345 | [0.215- | 0.677 | [0.548- | 0.222 | [0.12- |
| | | 0.767] | | 0.515] | | 0.806] | | 0.368] |
| Ridge | 0.669 | [0.576- | 0.343 | [0.202- | 0.67 | [0.542- | 0.217 | [0.12- |
| | | 0.762] | | 0.481] | | 0.798] | | 0.389] |
| Lasso | 0.668 | [0.575- | 0.348 | [0.212- | 0.676 | [0.544- | 0.226 | [0.116- |
| | | 0.762] | | 0.508] | | 0.808] | | 0.392] |
| PLS | 0.67 | [0.577- | 0.343 | [0.203- | 0.669 | [0.542- | 0.215 | [0.122- |
| | | 0.763] | | 0.491] | | 0.796] | | 0.374] |
| svmLin | 0.671 | [0.579- | 0.336 | [0.207- | 0.658 | [0.538- | 0.203 | [0.111- |
| | | 0.764] | | 0.493] | | 0.778] | | 0.335] |

### Combination of ADGRE3, C3AR1 and IL-10

TABLE 11

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.715 | [0.615- | 0.362 | [0.234- | 0.717 | [0.573- | 0.351 | [0.16- |
| | | 0.816] | | 0.507] | | 0.861] | | 0.63] |
| Ridge | 0.722 | [0.627- | 0.395 | [0.229- | 0.719 | [0.59- | 0.283 | [0.154- |
| | | 0.817] | | 0.545] | | 0.848] | | 0.517] |
| Lasso | 0.716 | [0.617- | 0.361 | [0.223- | 0.726 | [0.583- | 0.356 | [0.176- |
| | | 0.816] | | 0.51] | | 0.868] | | 0.622] |
| PLS | 0.721 | [0.626- | 0.407 | [0.25- | 0.717 | [0.587- | 0.279 | [0.131- |
| | | 0.817] | | 0.569] | | 0.847] | | 0.487] |
| svmLin | 0.727 | [0.63- | 0.398 | [0.231- | 0.724 | [0.593- | 0.313 | [0.162- |
| | | 0.823] | | 0.563] | | 0.855] | | 0.572] |

### Combination of ADGRE3, C3AR1 and OAS2

TABLE 12

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.67 | [0.573- | 0.38 | [0.23- | 0.655 | [0.524- | 0.208 | [0.101- |
| | | 0.767] | | 0.523] | | 0.785] | | 0.34] |
| Ridge | 0.669 | [0.569- | 0.398 | [0.262- | 0.665 | [0.531- | 0.219 | [0.126- |
| | | 0.769] | | 0.551] | | 0.799] | | 0.375] |
| Lasso | 0.663 | [0.564- | 0.381 | [0.231- | 0.661 | [0.53- | 0.214 | [0.114- |
| | | 0.763] | | 0.538] | | 0.791] | | 0.35] |
| PLS | 0.669 | [0.568- | 0.397 | [0.246- | 0.671 | [0.537- | 0.222 | [0.124- |
| | | 0.769] | | 0.544] | | 0.804] | | 0.378] |
| svmLin | 0.672 | [0.573- | 0.37 | [0.223- | 0.675 | [0.542- | 0.226 | [0.107- |
| | | 0.77] | | 0.531] | | 0.809] | | 0.374] |

### Combination of ADGRE3, CIITA and IL-10

TABLE 13

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.733 | [0.642- | 0.351 | [0.219- | 0.732 | [0.609- | 0.308 | [0.159- |
| | | 0.823] | | 0.513] | | 0.854] | | 0.588] |
| Ridge | 0.732 | [0.641- | 0.373 | [0.234- | 0.737 | [0.616- | 0.299 | [0.162- |
| | | 0.824] | | 0.535] | | 0.858] | | 0.558] |
| Lasso | 0.736 | [0.645- | 0.354 | [0.237- | 0.735 | [0.611- | 0.337 | [0.145- |
| | | 0.827] | | 0.519] | | 0.859] | | 0.604] |
| PLS | 0.731 | [0.639- | 0.372 | [0.233- | 0.738 | [0.616- | 0.298 | [0.151- |
| | | 0.822] | | 0.54] | | 0.859] | | 0.54] |
| svmLin | 0.727 | [0.633- | 0.376 | [0.24- | 0.744 | [0.619- | 0.344 | [0.154- |
| | | 0.821] | | 0.529] | | 0.868] | | 0.638] |

### Combination of ADGRE3, CIITA and OAS2

TABLE 14

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.664 | [0.571- | 0.323 | [0.195- | 0.692 | [0.553- | 0.257 | [0.123- |
| | | 0.757] | | 0.499] | | 0.832] | | 0.463] |
| Ridge | 0.663 | [0.568- | 0.333 | [0.197- | 0.692 | [0.551- | 0.262 | [0.131- |
| | | 0.758] | | 0.494] | | 0.834] | | 0.513] |
| Lasso | 0.657 | [0.563- | 0.323 | [0.202- | 0.668 | [0.518- | 0.244 | [0.141- |
| | | 0.75] | | 0.486] | | 0.818] | | 0.446] |
| PLS | 0.664 | [0.569- | 0.333 | [0.191- | 0.696 | [0.556- | 0.263 | [0.135- |
| | | 0.758] | | 0.484] | | 0.835] | | 0.493] |
| svmLin | 0.671 | [0.568- | 0.356 | [0.224- | 0.709 | [0.569- | 0.274 | [0.15- |
| | | 0.773] | | 0.516] | | 0.849] | | 0.515] |

### Combination of ADGRE3, OAS2 and IL-10

TABLE 15

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.715 | [0.615- | 0.362 | [0.244- | 0.717 | [0.573- | 0.351 | [0.156- |
| | | 0.816] | | 0.514] | | 0.861] | | 0.645] |
| Ridge | 0.715 | [0.62- | 0.391 | [0.25- | 0.744 | [0.618- | 0.319 | [0.157- |
| | | 0.81] | | 0.55] | | 0.871] | | 0.582] |
| Lasso | 0.715 | [0.615- | 0.358 | [0.236- | 0.72 | [0.577- | 0.344 | [0.164- |
| | | 0.815] | | 0.513] | | 0.862] | | 0.613] |
| PLS | 0.713 | [0.616- | 0.395 | [0.254- | 0.746 | [0.62- | 0.319 | [0.168- |
| | | 0.81] | | 0.545] | | 0.873] | | 0.594] |
| svmLin | 0.708 | [0.608- | 0.388 | [0.249- | 0.752 | [0.623- | 0.332 | [0.171- |
| | | 0.807] | | 0.549] | | 0.881] | | 0.612] |

### Combination of ADGRE3, IL-10, CD74 and CIITA

TABLE 16

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.732 | [0.641- | 0.353 | [0.243- | 0.732 | [0.611- | 0.305 | [0.168- |
| | | 0.823] | | 0.514] | | 0.854] | | 0.577] |
| Ridge | 0.725 | [0.632- | 0.375 | [0.235- | 0.752 | [0.618- | 0.328 | [0.172- |
| | | 0.818] | | 0.548] | | 0.886] | | 0.6] |
| Lasso | 0.738 | [0.645- | 0.379 | [0.241- | 0.74 | [0.618- | 0.33 | [0.165- |
| | | 0.83] | | 0.536] | | 0.863] | | 0.616] |
| PLS | 0.994 | [0.988- | 0.971 | [0.926- | 0.659 | [0.506- | 0.291 | [0.122- |
| | | 1] | | 0.997] | | 0.812] | | 0.578] |
| svmLin | 0.712 | [0.619- | 0.369 | [0.227- | 0.754 | [0.617- | 0.335 | [0.18- |
| | | 0.805] | | 0.546] | | 0.891] | | 0.61] |

### Combination of ADGRE3, C3AR1, CIITA and IL-10

TABLE 17

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.733 | [0.642- | 0.398 | [0.26- | 0.738 | [0.623- | 0.277 | [0.154- |
| | | 0.823] | | 0.57] | | 0.852] | | 0.519] |
| Ridge | 0.735 | [0.644- | 0.396 | [0.256- | 0.742 | [0.63- | 0.279 | [0.157- |
| | | 0.825] | | 0.556] | | 0.854] | | 0.494] |
| Lasso | 0.741 | [0.649- | 0.383 | [0.243- | 0.74 | [0.621- | 0.322 | [0.146- |
| | | 0.832] | | 0.549] | | 0.86] | | 0.583] |
| PLS | 0.731 | [0.641- | 0.393 | [0.247- | 0.739 | [0.625- | 0.277 | [0.14- |
| | | 0.821] | | 0.571] | | 0.853] | | 0.53] |
| svmLin | 0.732 | [0.635- | 0.397 | [0.242- | 0.732 | [0.605- | 0.318 | [0.165- |
| | | 0.828] | | 0.542] | | 0.86] | | 0.593] |

### Combination of ADGRE3, C3AR1, CIITA and OAS2

TABLE 18

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.686 | [0.595- | 0.34 | [0.214- | 0.709 | [0.587- | 0.241 | [0.126- |
| | | 0.777] | | 0.497] | | 0.832] | | 0.418] |
| Ridge | 0.683 | [0.591- | 0.362 | [0.228- | 0.699 | [0.575- | 0.236 | [0.131- |
| | | 0.775] | | 0.513] | | 0.824] | | 0.384] |
| Lasso | 0.676 | [0.584- | 0.36 | [0.218- | 0.697 | [0.569- | 0.238 | [0.131- |
| | | 0.769] | | 0.516] | | 0.824] | | 0.399] |
| PLS | 0.683 | [0.591- | 0.369 | [0.218- | 0.701 | [0.575- | 0.237 | [0.122- |
| | | 0.776] | | 0.539] | | 0.826] | | 0.419] |
| svmLin | 0.68 | [0.587- | 0.338 | [0.199- | 0.703 | [0.58- | 0.233 | [0.124- |
| | | 0.774] | | 0.508] | | 0.827] | | 0.373] |

### Combination of ADGRE3, C3AR1, OAS2 and IL-10

TABLE 19

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.728 | [0.631- | 0.432 | [0.287- | 0.734 | [0.605- | 0.299 | [0.167- |
| | | 0.825] | | 0.602] | | 0.864] | | 0.552] |
| Ridge | 0.726 | [0.631- | 0.421 | [0.251- | 0.735 | [0.606- | 0.297 | [0.149- |
| | | 0.821] | | 0.561] | | 0.864] | | 0.53] |
| Lasso | 0.726 | [0.629- | 0.4 | [0.253- | 0.726 | [0.59- | 0.337 | [0.15- |
| | | 0.824] | | 0.552] | | 0.863] | | 0.62] |
| PLS | 0.723 | [0.627- | 0.431 | [0.261- | 0.73 | [0.602- | 0.285 | [0.141- |
| | | 0.819] | | 0.594] | | 0.858] | | 0.56] |
| svmLin | 0.727 | [0.63- | 0.414 | [0.259- | 0.744 | [0.618- | 0.318 | [0.172- |
| | | 0.824] | | 0.573] | | 0.871] | | 0.595] |

### Combination of ADGRE3, OAS2, CIITA and IL-10

TABLE 20

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.727 | [0.633- | 0.347 | [0.234- | 0.729 | [0.596- | 0.36 | [0.149- |
| | | 0.822] | | 0.518] | | 0.862] | | 0.678] |
| Ridge | 0.727 | [0.634- | 0.387 | [0.239- | 0.75 | [0.632- | 0.306 | [0.145- |
| | | 0.82] | | 0.536] | | 0.868] | | 0.567] |
| Lasso | 0.738 | [0.646- | 0.381 | [0.246- | 0.743 | [0.623- | 0.316 | [0.164- |
| | | 0.83] | | 0.557] | | 0.863] | | 0.575] |
| PLS | 0.726 | [0.632- | 0.389 | [0.242- | 0.746 | [0.627- | 0.3 | [0.16- |
| | | 0.819] | | 0.552] | | 0.865] | | 0.587] |
| svmLin | 0.73 | [0.636- | 0.388 | [0.248- | 0.751 | [0.629- | 0.33 | [0.169- |
| | | 0.824] | | 0.551] | | 0.873] | | 0.623] |

### Combination of ADGRE3, C3AR1, CIITA, IL-10 and OAS2

TABLE 21

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.731 | [0.639- | 0.386 | [0.232- | 0.746 | [0.629- | 0.281 | [0.146- |
| | | 0.823] | | 0.56] | | 0.863] | | 0.5] |
| Ridge | 0.736 | [0.644- | 0.406 | [0.254- | 0.747 | [0.631- | 0.286 | [0.149- |
| | | 0.828] | | 0.578] | | 0.863] | | 0.509] |
| Lasso | 0.737 | [0.644- | 0.379 | [0.243- | 0.744 | [0.621- | 0.347 | [0.173- |
| | | 0.83] | | 0.545] | | 0.868] | | 0.621] |
| PLS | 0.733 | [0.641- | 0.414 | [0.263- | 0.746 | [0.63- | 0.284 | [0.147- |
| | | 0.825] | | 0.573] | | 0.863] | | 0.513] |
| svmLin | 0.737 | [0.643- | 0.398 | [0.249- | 0.75 | [0.627- | 0.326 | [0.155- |
| | | 0.831] | | 0.564] | | 0.872] | | 0.587] |

### Combination of ADGRE3, C3AR1, CD177. IL10, CIITA, IL1R2 and OAS2

TABLE 22

| | Train | | | | Test | | | |
|---|---|---|---|---|---|---|---|---|
| | | AUROC | | AUPRC | | AUROC | | AUPRC |
| Model | AUROC | 95% CI | AUPRC | 95% CI | AUROC | 95% CI | AUPRC | 95% CI |
| Elasticnet | 0.733 | [0.642- | 0.349 | [0.223- | 0.732 | [0.607- | 0.324 | [0.147- |
| | | 0.824] | | 0.522] | | 0.857] | | 0.59] |
| Ridge | 0.733 | [0.646- | 0.377 | [0.243- | 0.738 | [0.612- | 0.314 | [0.167- |
| | | 0.82] | | 0.535] | | 0.865] | | 0.565] |
| Lasso | 0.717 | [0.617- | 0.359 | [0.23- | 0.721 | [0.577- | 0.359 | [0.186- |
| | | 0.816] | | 0.521] | | 0.864] | | 0.635] |
| PLS | 0.727 | [0.639- | 0.381 | [0.238- | 0.728 | [0.597- | 0.3 | [0.137- |
| | | 0.815] | | 0.554] | | 0.859] | | 0.553] |
| svmLin | 0.734 | [0.639- | 0.41 | [0.258- | 0.732 | [0.603- | 0.31 | [0.149- |
| | | 0.829] | | 0.556] | | 0.861] | | 0.581] |

Finally, the models were applied to a signature containing the ADGRE3 biomarker and the eight additional biomarkers C3AR1, CD177, IL10, CIITA, IL1R2, OAS2, CD74 and TDRD9:

TABLE 23

| | AUROC_{training} | AUPRC_{training} | AUROCₜₑₛₜ | AUPRCₜₑₛₜ |
|---|---|---|---|---|
| Model | [95% CI] | [95% CI] | [95% CI] | [95% CI] |
| Elastic Net | 0.715 | 0.361 | 0.721 | 0.380 |
| | [0.575-0.844] | [0.243-0.524] | [0.493-0.938] | [0.171-0.662] |
| Ridge | 0.737 | 0.406 | 0.751 | 0.326 |
| | [0.612-0.859] | [0.257-0.584] | [0.575-0.927] | [0.164-0.558] |
| Lasso | 0.754 | 0.402 | 0.748 | 0.346 |
| | [0.630-0.874] | [0.256-0.576] | [0.554-0.932] | [0.168-0.620] |
| PLS | 0.732 | 0.406 | 0.744 | 0.312 |
| | [0.605-0.853] | [0.256-0.579] | [0.567-0.924] | [0.156-0.566] |
| svmLin | 0.744 | 0.431 | 0.764 | 0.431 |
| | [0.600-0.881] | [0.278-0.610] | [0.536-0.960] | [0.214-0.720] |

All of the performance thereby demonstrates that measuring the ADGRE3 expression level, particularly in combination with measuring the expression level of one or more particular additional genes, makes it possible to determine whether a patient infected with a respiratory virus such as SARS-Cov-2 has an increased risk of death.

Preferably, the most advantageous combinations of biomarkers for predicting an increased risk of death in a subject infected with a respiratory virus are those for which the majority of the training models make it possible to obtain an AUROC of at least 0.7. Moreover, since mortality in the test dataset is 17%, it is particularly advantageous to also target those combinations for which the majority of models make it possible to obtain an AUPRC of at least 0.3, preferably at least 0.32.

### BIBLIOGRAPHICAL REFERENCES

C. Huang, et al., “Clinical features of patients infected with 2019 novel coronavirus in Wuhan, China” Lancet, 2020, 395, 497, https://doi.org/10.1016/S0140-6736 (20) 30183-5;
E. Z. Ong, et al., “A Dynamic Immune Response Shapes COVID-19 Progression” Cell Host Microbe, 2020, 27 (6), 879, https://doi.org/10.1016/j.chom.2020.03.021;
F. Venet et al., “Longitudinal assessment of IFN-I activity and immune profile in critically ill COVID-19 patients with acute respiratory distress syndrome”; Crit Care, 2021, 25 (1), 140, https://doi.org/10.1186/s13054-021-03558-w;
Hadjadj, J. et al. “Impaired type I interferon activity and inflammatory responses in severe COVID-19 patients”. Science 369, 718-724 (2020);
M. S. Abers, et al., “An immune-based biomarker signature is associated with mortality in COVID-19 patients” JCI Insight, 2021, 6 (1): e144455, https://doi.org/10.1172/jci.insight.144455;
Guardela et al., “50-gene risk profiles in peripheral blood predict COVID-19 outcomes: A retrospective, multicenter cohort study” EBioMedecine, 2021, https://doi.org/10.1016/j.ebiom.2021.103439;
Y. Levy et al., “CD177, a specific marker of neutrophil activation, is associated with coronavirus disease 2019 severity and death” Iscience, 2021, 24 (7): 102711 DOI: 10.1016/j.isci.2021.102711;
G. Canedo-Marroquín et al., “SARS-COV-2: Immune Response Elicited by Infection and Development of Vaccines and Treatments” Front. Immunol, 2020, 11:569760; DOI 10.3389/fimmu.2020.569760;
Chawla, N. V et al., SMOTE: synthetic minority over-sampling technique. J. Artif. Int. Res. 16, 321-357 (2002);
Ozenne B. et al, “The precision-recall curve overcame the optimism of the receiver operating characteristic curve in rare diseases”. J Clin Epidemiol 68, 855-859, doi: 10.1016/j.jclinepi.2015.02.010 (2015);
Greenwell, et al., “A Simple and Effective Model-Based Variable Importance Measure”. ArXiv abs/1805.04755 (2018);

1. An in vitro or ex vivo method for determining the risk of death of a subject infected with a respiratory virus, said method comprising the following steps:
a) measuring the expression level of the ADGRE3 gene in a biological sample from said subject,
b) comparing the expression level of the ADGRE3 gene measured in step a) with a predetermined reference value.

2. The method as claimed in claim 1, characterized in that the respiratory virus is SARS-COV-2 or a variant thereof.

3. The method as claimed in claim 1 or 2, characterized in that it also comprises a step c) of concluding that there is an increased risk of death of said subject on the basis of the result of the comparison when under-expression of said gene at the mRNA level is demonstrated.

4. The method as claimed in one of claims 1 to 3, characterized in that the reference value corresponds to the average ADGRE3 expression level obtained from biological samples originating from a population of subjects who are not infected with any respiratory virus, or to the average ADGRE3 expression level obtained from biological samples originating from a population of subjects who are infected with a respiratory virus and who are known to have survived following infection, particularly in the 28 days following admission to a healthcare facility.

5. The method as claimed in one of claims 1 to 4, characterized in that it further comprises a step of measuring the expression of at least one other additional gene selected from C3AR1, CD177, OAS2, CIITA, IL-10, IL1R2, CD74, TDRD9 and combinations thereof in the biological sample from said subject.

6. The method as claimed in claim 5, characterized in that it further comprises a step of measuring the expression of at least one other additional gene selected from C3AR1, CD177, OAS2, CIITA, IL-10, IL1R2 and combinations thereof, and preferably from C3AR1, CIITA, CD177, IL-10 and combinations thereof, in the biological sample from said subject.

7. The method as claimed in one of claims 1 to 6, characterized in that the expression of said gene(s) is measured at the mRNA level.

8. The method as claimed in one of claims 1 to 7, characterized in that the biological sample is a blood sample, preferably a total blood sample.

9. The method as claimed in one of claims 1 to 8, characterized in that the expression is measured by amplification, sequencing or hybridization.

10. The method as claimed in one of claims 1 to 9, characterized in that the expression is measured by amplification using RT-PCR, preferably RT-qPCR.

11. The method as claimed in one of claims 1 to 10, characterized in that the expression is normalized in relation to the expression of one or more housekeeping genes.

12. A kit for the in vitro or ex vivo measurement of the expression of ADGRE3 in a biological sample, comprising means for determining the ADGRE3 expression level in said sample, said means being selected from amplification primers or probes.

13. The kit as claimed in claim 12, characterized in that it comprises a positive control sample calibrated to contain the quantity of ADGRE3 that corresponds to the quantity or concentration representative of the expression level, measured at the mRNA level, in a pool of samples from subjects who are not infected with a respiratory virus, and/or a negative control sample calibrated to contain the quantity of ADGRE3 that corresponds to the average quantity measured in a pool of samples from subjects who did not survive following infection with a respiratory virus.

14. The kit as claimed in claim 12 or 13, characterized in that it comprises means for determining the expression level of at least one other additional gene selected from C3AR1, CD177, OAS2, CIITA, IL-10, IL1R2, CD74, TDRD9 and combinations thereof, preferably selected from C3AR1, CD177, CIITA, IL-10, IL1R2 and combinations thereof, and more preferably selected from C3AR1, CIITA, IL-10 and combinations thereof.

15. The use of the kit as claimed in one of claims 12 to 14 for determining the risk of death, preferably the risk of death in the 28 days following admission to a healthcare facility, of a subject infected with a respiratory virus such as SARS-CoV-2 or a variant thereof.
